(19) United States
(12) Patent Application Publication
(10) Pub. No.: US 2012/0076734 A1
(43) Pub. Date: Mar. 29, 2012

(54) RADIOPAQUE ANTIBIOTIC DENTAL PASTE AND USES THEREOF

(76) Inventor: Jacob Olson, Menomonee Falls, WI (US)

(21) Appl. No.: 13/219,181

(22) Filed: Aug. 26, 2011

Related U.S. Application Data
(60) Provisional application No. 61/383,232, filed on Sep. 15, 2010.

(51) Int. Cl. A61K 49/04; A61K 31/7056; A61K 6/00; A61P 23/00; A61P 25/04; A61K 31/545; A61P 31/00

(52) U.S. Cl. 424/9.4; 514/200; 514/24

(57) ABSTRACT

Compositions useful for disinfecting a dental wound are described. The compositions include an active ingredient comprising one or more antibiotics, a radiopaque substance, and optionally a pharmaceutically acceptable excipient. Methods for treating dental wound sites with the compositions are also described.

### CROSS REFERENCE TO RELATED APPLICATION

This application is a non-provisional application of U.S. provisional application No. 61/383,232 filed Sep. 15, 2010, entitled “RADIOPAQUE ANTIBIOTIC DENTAL PASTE AND USES THEREOF”, which is hereby incorporated by reference in its entirety.

### BACKGROUND OF THE INVENTION

Dental treatments, including treatments for cavities, endodontic treatments and treatments for periodontitis, require the use of disinfectant compositions, such as antibiotic mixtures. During an endodontic treatment, for example, the regeneration of pulp and pulp revascularization will occur only in the absence of intracanal infections, and disinfection of the root canal system is a key factor in the success of such treatment modalities. Typical disinfection methods involve aggressive procedures such as cleaning with an endodontic file.

In children's teeth, where the root development is immature, the infected space cannot be cleaned using typical endodontic procedures. For such cases, disinfectants containing a mixture of antibiotics have been used and found to promote revascularization of the dental pulp. Some antibiotics used as disinfectants additionally cause discoloration of the teeth and gums, and some antibiotic mixtures have little shelf-life or stability if mixed and stored prior to treatment.

During root canals, antibiotic treatment is combined with calcium hydroxide or mineral aggregates to initiate healing and hard tissue formation. The calcium hydroxide or mineral aggregate is later washed out prior to filling of the root canal and washing out is evaluated by radiography. Without the use of radiopaque substances, it becomes difficult to locate and determine whether the antibiotics or other treatment components, such as calcium hydroxide or mineral aggregates, are actually in the pulpal space or apex of the tooth. A need therefore exists for a pharmaceutical composition that can be used to treat dental wound sites with disinfectant properties that is sufficiently radiopaque to allow visualization of the composition in the apical space during treatment.

### SUMMARY OF THE INVENTION

A composition and methods for disinfecting an endodontic wound site formed from an injury, periodontal disease or disorder, or during dental treatment with a composition of the invention is described. In an embodiment, the wound site comprises damage to the pulp of a tooth. The composition includes an active ingredient comprising one or more antibiotics, a radiopaque substance, and a pharmaceutically acceptable carrier and/or excipient. The composition can optionally include an anesthetic agent, analgesic agent, or combination thereof. In an aspect, the radiopaque substance is iodoform. In another aspect, the carrier or excipient is glycerin, polyethylene glycol (PEG), or a mixture of PEG with different molecular weights. The composition of the invention can be premixed and stored for a period of time before treatment, or the components can be provided in a kit form and mixed together to form the composition of the invention as needed immediately prior to use.

The composition includes an active ingredient comprising a mixture of one or more antibiotics. In an embodiment, one or more of the antibiotics is selected from nitroimidazole, fluoroquinolone, cephalosporin or lincosamide antibiotics. In an aspect, the nitroimidazole is metranidazole, the fluoroquinolone is ciprofloxacin, the lincosamide is clindamycin and the cephalosporin is cefaclor. Preferably, the composition does not include an antibiotic which is known to stain the teeth or gums. One example of such an antibiotic is minocycline.

Methods for disinfecting a dental wound site with a composition of the invention are also described. The methods generally include applying a composition of the invention to a dental wound and then radioimaging the dental wound site to visualize the placement of the composition in the dental wound site.

It is to be understood that both the foregoing general description and the following detailed description are exemplary and explanatory only and are not restrictive of the invention, as claimed. The accompanying drawings, which are incorporated in and constitute a part of this specification, illustrate an example embodiment of the invention and together with the description, serve to explain the principles of the invention.

### BRIEF DESCRIPTION OF THE DRAWINGS

FIG. 1 shows a radiograph of a sample of a composition of the invention demonstrating the radiopacity of the composition.

### DETAILED DESCRIPTION OF THE PREFERRED EMBODIMENT

The invention described herein provides a composition and methods for disinfecting a dental wound site. The dental wound site can be due to injury, a periodontal disorder or disease, or dental treatment. The periodontal disease or disorder includes, but is not limited to, apical periodontitis, pulpal necrosis as a result of caries or trauma, and the like. The dental wound site includes, but is not limited to a tooth or root canal. The tooth can be a mature or immature permanent tooth.

The composition described herein is a disinfectant composition for dental wound sites. The composition comprises an active ingredient comprising one or more antibiotics, a radiopaque substance, and optionally a pharmaceutically acceptable carrier and/or excipient. Compositions of the invention can optionally include one or more anesthetic agents, one or more analgesic agents, or a combination thereof.

In a dental wound, regeneration of the pulpal tissue of a tooth generally halts at the level where bacteria are found. The composition provides effective antibiotic action or disinfectant action at the wound site, while also providing an easy method for determining if the product has been placed correctly in the dental wound during treatment. The antibiotic action of the composition provides a suitable disinfected environment at the site of the dental wound which promotes regeneration and/or revascularization of the pulp of the affected tooth.

The composition described herein includes an active ingredient comprising a mixture of one or more antibiotics, preferably a mixture of at least three antibiotics. The term “antibiotic” refers to a substance or compound capable of eliminating or inhibiting the growth of microorganisms or pathogens. The compounds and methods of the present invention include antibiotics that are bactericidal or bacteriostatic. Bactericidal antibiotics include, without limitation, penicillins, cephalosporins, polymixins, quinolones, nitroimidazoles or sulfonamides, for example. Bacteriostatic antibiotics include, without limitation, aminoglycosides, lincosamides, macrolides and tetracyclines, for example. Antibiotics useful in the compositions and methods of the present invention include broad spectrum and narrow-spectrum agents, targeting either Gram-negative or Gram-positive microorganisms.

The composition can include a combination of one, two, three, four, or five or more different antibiotics. Preferably, the composition includes at least three antibiotics. In an embodiment, at least one of the antibiotics is a bacteriostatic agent. In another embodiment, at least one of the antibiotics is a bactericidal agent. In yet another embodiment, a combination of antibiotics is selected such that the composition includes both bactericidal agents and bacteriostatic agents. Bactericidal agents include, without limitation, penicillins, cephalosporins, quinolones, nitroimidazoles, and the like. Bacteriostatic agents include, without limitation, lincosamides, macrolides, tetracyclines, aminoglycosides and the like.

In an embodiment, the antibiotic component comprises one or more of a penicillin, sulfonamide, nitroimidazole, cephalosporin, lincosamide, or quinolone. Penicillins include, without limitation, amoxicillin, augmentin and the like. Sulfonamides include, without limitation, sulfamethoxazole, combinations thereof with trimethoprim, and the like. Nitroimidazoles include, without limitation, metronidazole, tinidazole, nimorazole and the like. Cephalosporins include without limitation, cefaclor, cefonicid, cefprozil, cefuroxime, cefuzonam, cefmetazole, cefoxitin, cefotetan and the like. Quinolones include fluoroquinolones such as ciprofloxacin, moxifloxacin, levofloxacin, and the like. Lincosamides include, without limitation, lincomycin, clindamycin and the like, for example. In another embodiment the compositions comprises a mixture of nitromidazole, cephalosporin or lincosamide, and a quinolone. In yet another embodiment, the composition comprises a mixture of metronidazole, ciprofloxacin, and cefaclor or clindamycin.

The active ingredient comprises about 1 to about 100 wt % of the composition, In an embodiment, the active ingredient comprises about 1 to about 50 wt %, about 1 to about 45 wt %, about 1 to about 40 wt %, about 1 to about 35 wt %, about 1 to about 30 wt %, about 1 to about 25 wt %, about 1 to about 20%, about 1 to about 19 wt %, about 1 to about 18 wt %, about 1 to about 17 wt %, about 1 to about 16 wt %, about 1 to about 15 wt %, about 1 to about 14 wt %, about 1 to about 13 wt %, about 1 to about 12 wt %, about 1 to about 11 wt %, about 1 to about 10 wt %, about 1 to about 9 wt %, about 1 to about 8 wt %. about 1 to about 7 wt %, about 1 to about 6 wt %, about 1 to about 5 wt %, about 1 to about 4 wt %, about 1 to about 3 wt %, or about 1 to about 2 wt % of the composition. In another embodiment, the active ingredient comprises about 5 to about 95 wt %, about 5 to about 90 wt %, about 5 to about 85 wt %, about 5 to about 80 wt %, about 5 to about 75 wt %, about 5 to about 70 wt %, about 5 to about 65 wt %, about 5 to about 60 wt %, about 5 to about 55 wt %, about 5 to about 50 wt %, about 5 to about 45 wt %, about 5 to about 40 wt %, about 5 to about 35 wt %, about 5 to about 30 wt %, about 5 to about 25 wt %, about 5 to about 20 wt %, about 5 to about 15 wt %, or about 5 to about 10 wt % of the composition. In yet another embodiment, the active ingredient comprises about 10 to about 95 wt %, about 10 to about 90 wt %, about 10 to about 85 wt %, about 10 to about 80 wt %, about 10 to about 75 wt %, about 10 to about 70 wt %, about 10 to about 65 wt %, about 10 to about 60 wt %, about 10 to about 55 wt %, about 10 to about 50 wt %, about 10 to about 45 wt %, about 10 to about 40 wt %, about 10 to about 35 wt %, about 10 to about 30 wt %, about 10 to about 25 wt %, about 10 to about 20 wt %, or about 10 to about 15 wt % of the composition.

In an embodiment, the active ingredient comprises about 10 to about 50 wt % nitroimidazole, about 10 to about 50 wt % cephalosporin or lincosamide, and about 1 to about 30 wt % quinolone. In another embodiment, the active ingredient comprises about 30 to about 50 wt % nitroimidazole, about 30 to about 50 wt % cephalosporin or lincosamide, and about 5 to about 30 wt % quinolone. In yet another embodiment, the active ingredient comprises about 40 to about 50 wt % nitroimidazole, about 40 to about 50 wt % cephalosporin or lincosamide, and about 5 to about 20 wt % quinolone.

In an embodiment, the active ingredient comprises about 10 to about 50 wt % metronidazole, about 10 to about 50 wt % ciprofloxacin, and about 1 to about 30 wt % cefaclor or clindamycin. In another embodiment, the active ingredient comprises about 30 to about 50 wt % metronidazole, about 30 to about 50 wt % ciprofloxacin, and about 5 to about 30 wt % cefaclor or clindamycin. In yet another embodiment, the active ingredient comprises about 40 to about 50 wt % metronidazole, about 40 to about 50 wt % ciprofloxacin, and about 5 to about 20 wt % cefaclor or clindamycin.

In an embodiment, the composition comprises a mixture of antibiotics in the following proportion: about 2 to about 3 parts by weight of a nitroimidazole, about 0.5 to about 1 part by weight of a quinolone, and about 2 to about 3 parts by weight of a cephalosporin or a lincosamide. In another embodiment, the composition comprises a mixture of antibiotics in the following proportion: about 3 parts by weight of a nitroimidazole, about 1 part by weight of a quinolone, and about 3 parts by weight of a cephalosporin or a lincosamide. In another embodiment, the composition comprises from about 0.5 to about 25 wt % quinolone, about 0.5 to about 25 wt % nitroimidazole, about 0.5 to about 25 wt % cephalosporin or lincosamide antibiotics; or about 5 to about 50 wt % nitroimidazoles, about 5 to about 50 wt % quinolone, and about 5 to about 25 wt % cephalosporin or lincosamide antibiotics; or about 25 to about 50 wt % nitroimidazoles, or about 25 to about 50 wt % quinolone, and about 10 to about 25 wt % cephalosporin or lincosamide antibiotics. In yet another embodiment, the composition comprises about 0.4 to about 3.2 wt % quinolone, about 2 to about 8 wt % nitroimidazole, and about 2 to about 8 wt % cephalosporin or lincosamide; preferably about 0.8 to about 2.4 wt % quinolone, about 4 to about 6 wt % nitroimidazole, and about 4 to about 6 wt % cephalosporin or lincosamide; more preferably about 2 wt % quinolone, about 5 wt % nitroimidazole, and about 5 wt % cephalosporin or lincosamide.

In an embodiment, the composition comprises a mixture of three antibiotics with the antibiotics in the following proportion: about 3 parts by weight of metronidazole, about 1 part by weight of ciprofloxacin, and about 3 parts by weight of cefaclor or clindamycin. In an aspect, the composition comprises about 0.5 to about 25 wt % ciprofloxacin, about 0.5 to about 25 wt % metranidazole, and about 0.5 to about 25 wt % of either cefaclor or clindamycin; or about 5 to about 50 wt % metranidazole, about 5 to about 50 wt % ciprofloxacin, and about 5 to about 25 wt % cefaclor or clindamycin antibiotics; or about 25 wt % to about 50 wt % metranidazole, about 25 wt % to about 50 wt % ciprofloxacin, and about 10 wt % to about 25 wt % cefaclor or clindamycin antibiotics. In another aspect, the composition comprises about 0.4 to about 3.2 wt % ciprofloxacin, about 2 to about 8 wt % metronidazole, and about 2 to about 8 wt % cefaclor or clindamycin; preferably about 0.8 to about 2.4 wt % ciprofloxacin, about 4 to about 6 wt % metronidazole, and about 4 to about 6 wt % cefaclor or clindamycin; more preferably about 2 wt % ciprofloxacin, about 5 wt % metronidazole, and about 5 wt % cefaclor or clindamycin.

The composition of the invention comprises a substance for producing radiopacity. The term “radiopaque” or “radiopacity” refers to the ability of a substance to block transmission or passage of x-rays through the substance. Typically, radiopaque substances are dense materials that provide a white appearance in x-rays and radiographs. Radiopaque substances are therefore useful in tracking or locating an implanted device or treatment modality during various different types of intervention therapies, including dental treatment. Typical radiopaque substances include, without limitation, calcium hydroxide, barium sulfate, barium particles, diatrizoate, acetrizoate and salts thereof, ethiodized oil, organoioidide compounds, including without limitation, iodoform, iobenzamic acid, iocarmic acid, iocetamic acid, iodipamide, iodixanol, iodized oil, iodophthalein, iodopyracet, ioglycamic acid, iohexol, iomeglamic acid, ioversol, and the like, thorium oxide, trypanoate sodium, and other metals, such as bismuth, uranium, antimony, tungsten and the like, either in their salt forms or complexed with a polymer. The radiopaque substance can be an active ingredient that has anti-microbial activity. One example of such a radiopaque substance is iodoform.

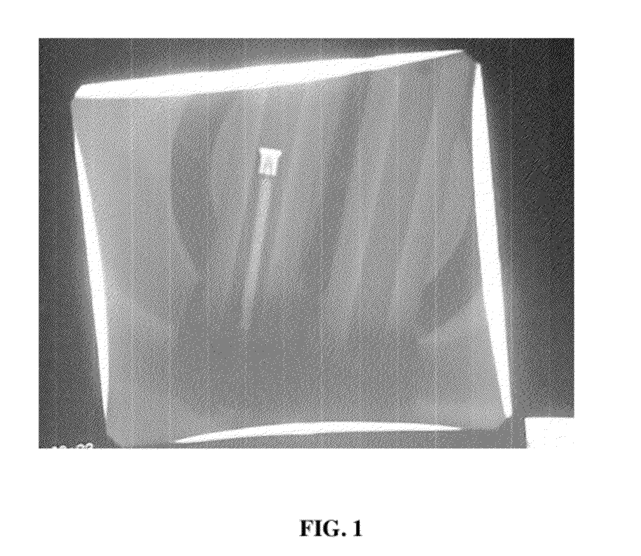

In an embodiment, the radiopaque substance comprises iodoform (CHI₃). In an embodiment, the iodoform is in a paste or powder form. The radiopaque substance allows for radioimaging of the composition such that radioimaging of a dental wound site treated with the composition allows for the visualization of the composition on an x-ray radiograph, thereby confirming that the composition is properly placed in the dental wound and occupies, for example, the root canal system or the apical space in the tooth. This is further demonstrated in FIG. 1, which shows an x-ray radiograph of a sample of a composition of the invention. As can be seen in FIG. 1, the composition of the invention is radiopaque and provides an easy tool for identifying where the composition has been placed during dental treatment.

In an embodiment, the radiopaque substance comprises about 1 to about 50 wt %, about 1 to about 40 wt %, about 1 to about 30 wt %, about 1 to about 25 wt %, about 1 to about 20 wt %, about 1 to about 19 wt %, about 1 to about 18 wt %, about 1 to about 17 wt %, about 1 to about 16 wt %, about 1 to about 15 wt %, about 1 to about 14 wt %, about 1 to about 13 wt %, about 1 to about 12 wt %, about 1 to about 11 wt %, about 1 to about 10 wt %, about 1 to about 9 wt %, about 1 to about 8 wt %. about 1 to about 7 wt %, about 1 to about 6 wt %, about 1 to about 5 wt %, about 1 to about 4 wt %, about 1 to about 3 wt %, or about 1 to about 2 wt % of the composition. In another embodiment, the radiopaque substance comprises about 5 to about 50 wt %, about 5 to about 40 wt %, about 5 to about 30 wt %, about 5 to about 25 wt %, about 5 to about 20 wt %, about 5 to about 15 wt %, or about 5 to about 10 wt % of the composition. In yet another embodiment, the composition comprises about 2 to about 8 wt % radiopaque substance, about 4 to about 6 wt % radiopaque substance, or about 5 wt % radio opaque substance.

In an embodiment, the composition comprises nitroimidazole, quinolone, cephalosporin or lincosamide, and a radiopaque substance. In an aspect, the composition about 2 to about 3 parts by weight of a nitroimidazole, about 0.1 to about 1 part by weight of a quinolone, about 2 to about 3 parts by weight of a cephalosporin or a lincosamide, and about 2 to about 3 parts by weight of a radiopaque substance. In another aspect, the composition comprises from about 0.5 to about 30 wt % quinolone, about 0.5 to about 30 wt % nitroimidazole, about 0.5 to about 30 wt % cephalosporin or lincosamide, and about 0.5 to about 30 wt % radiopaque substance. In another aspect, the composition comprises from about 0.5 to about 10 wt % quinolone, about 0.5 to about 30 wt % nitroimidazole, about 0.5 to about 30 wt % cephalosporin or lincosamide, and about 0.5 to about 30 wt % radiopaque substance. In another aspect, the composition comprises about 10 to about 30 wt % nitroimidazoles, about 10 to about 30 wt % quinolone, about 3 to about 15 wt % cephalosporin or lincosamide, and about 10 to about 30 wt % radiopaque substance. In another aspect, the composition comprises about 5 to about 20 wt % nitroimidazoles, or about 5 to about 20 wt % quinolone, about 1 to about 10 wt % cephalosporin or lincosamide antibiotics, and about 5 to about 20 wt % radiopaque. In yet another aspect, the composition comprises about 0.4 to about 3.2 wt % quinolone, about 2 to about 8 wt % nitroimidazole, about 2 to about 8 wt % cephalosporin or lincosamide, and about 2 to about 8 wt % radiopaque substance; or about 0.8 to about 2.4 wt % quinolone, about 4 to about 6 wt % nitroimidazole, about 4 to about 6 wt % cephalosporin or lincosamide, and about 4 to about 6 wt % radiopaque substance; or about 2 wt % quinolone, about 5 wt % nitroimidazole, about 5 wt % cephalosporin or lincosamide, and about 5 wt % radiopaque substance.

In an embodiment, the composition comprises metronidazole, ciprofloxacin, cefaclor or clindamycin, and a radiopaque substance. In an aspect, the composition comprises about 2 to about 3 parts by weight of metronidazole, about 2 to about 3 parts by weight of cefaclor or clindamycin, about 0.1 to about 1 part by weight of ciprofloxacin, and about 2 to about 3 parts by weight iodoform. In another aspect, the composition comprises from about 0.5 to about 30 wt % metronidazole, about 0.5 to about 30 wt % cefaclor or clindamycin, about 0.5 to about 30 wt % ciproflaxin, and about 0.5 to about 30 wt % radiopaque substance. In another aspect, the composition comprises from about 0.5 to about 30 wt % metronidazole, about 0.5 to about 30 wt % cefaclor or clindamycin, about 0.5 to about 10 wt % ciproflaxin, and about 0.5 to about 30 wt % radiopaque substance. In another aspect, the composition comprises about 10 to about 30 wt % metronidazole, about 10 to about 30 wt % cefaclor or clindamycin, about 3 to about 15 wt % ciprofloxacin, and about 10 to about 30 wt % radiopaque substance. In another aspect, the composition comprises about 5 to about 20 wt % metronidazole, about 5 to about 20 wt % cefaclor or clindamycin, about 1 to about 10 wt % ciprofloxacin, and about 5 to about 20 wt % radiopaque. In yet another aspect, the composition comprises about 0.4 to about 3.2 wt % ciprofloxacin, about 2 to about 8 wt % metronidazole, about 2 to about 8 wt % cefaclor or clindamycin, and about 2 to about 8% iodoform; or about 0.8 to about 2.4 wt % ciprofloxacin, about 4 to about 6 wt % metronidazole, about 4 to about 6 wt % cefaclor or clindamycin, and about 4 to about 6 wt % iodoform; or preferably about 2 wt % ciprofloxacin, about 5 wt % metronidazole, about 5 wt % cefaclor or clindamycin, and about 5 wt % iodoform.

The composition can optionally include one or more anesthetic agents, one or more analgesic agents, or a combination thereof. Preferably, the anesthetic agent is a topical anesthetic agent. Examples of topical anesthetic agents include, but are not limited to, novocain, nesacaine, xylocaine, lidocaine, mepivacaine, polocaine, carbocaine, marcaine, bupivacaine, etidocaine, prilocaine, citanest, duranest, ropivacaine, and naropin. Examples of analgesic agents include, but are not limited to, salicylates, acetaminophen, non-steroidal anti-inflammatory agents (NSAID). Examples of NSAIDs include, but are not limited to, ibuprofen, naproxen, naproxen sodium, celecoxib, odiclofenae, diflunisal etodolac, fenoprofen, ketorolac, mefenamic acid, oxaprozin, sulindac, tolmetin, piroxican, indomethacin, etodolac, meloxicam, ketoprofen, and nabumetone. Dosage of the anesthetic agent and/or analgesic agent is dependent on the procedure, degree of anesthesia or pain relief required, and individual patient circumstances as routinely determined by one of skill in the art. See, for example, Toxicity, Local Anesthetics by Kapitanayn and Tarabar at http://emedicine.medscape.com/article/1844551-overview and the United States Food and Drug Administration's (FDA) Medication Guide for NSAIDs at http://www.fda.gov/downloads/Drugs/DrugSafety/PostmarketDrugSafetyInformationforPatientsandProviders/UCM106241.pdf.

The composition can include a pharmaceutically acceptable carrier and/or excipient. The terms “excipient” and “carrier” are used interchangeably herein and denote pharmaceutically inert substances that can be used as a carrier for the active ingredient(s). In an aspect, the excipients or carriers used with the compositions of the present invention are binders. Binders are used to hold ingredients in a composition together and prevent segregation of components in a formulation. In an aspect, the excipients used in the composition of the present invention are, without limitation, saccharides, including disaccharides such as sucrose and lactose, polysaccharides such as starches, cellulose, and cellulose ethers, and sugar alcohols such as xylitol, sorbitol and mannitol, for example. Excipients may also be proteins such as gelatin, synthetic polymers, such as polyvinyl pyrrolidone (PVP) and polyethylene glycol (PEG) and the like. Excipients may be used in solid, liquid or other forms. Where the excipient is a binder, it is used in solution form (where the excipient is first dissolved in a solvent) or in dry form (where the excipient is added to a solid or dry blend of active ingredients). Examples of solution binders include, without limitation, gelatin, cellulose, cellulose derivatives, polyvinylpyrrolidone, starch, sucrose, polyethylene glycol (PEG) and the like. Examples of dry binders include, without limitation, cellulose, methyl cellulose, polyvinylpyrrolidone (PVP), polyethylene glycol (PEG) and the like.

In an embodiment, the pharmaceutically acceptable excipient or carrier comprises from about 0 to 99 wt %, about 10 wt % to 95 wt %, from about 20 wt % to 95 wt %, about 50 wt % to about 95 wt %, about 70 to about 95 wt %, about 75 to about 95 wt %, about 80 to about 95 wt %, about 85 to about 95 wt %, about 90 to about 95 wt %, or about 80 to about 85 wt % of the composition. In another embodiment, the pharmaceutically acceptable excipient or carrier comprises from about 0 to about 50 wt %, about 1 to about 40 wt %, about 1 to about 30 wt %, about 1 to about 20 wt %, about 1 to about 15 wt %, about 1 to about 10 wt %, or about 1 to about 5 wt % of the composition.

In an embodiment, the excipient or carrier comprises glycerin, propylene glycol, macrogel ointment, PEG, or a combination thereof. The PEG may have a molecular weight of about 300 or more. In another embodiment, the excipient or carrier comprises a mixture of PEGs with different molecular weights, ranging from 300 to 10,000, from 300 to 5,000, from 300 to 1,500, from 300 to 1,000, and the like. In yet another embodiment, the excipient or carrier comprises a two part system comprising a mixture of PEG 300 and PEG 1450. In an aspect, the ratio of PEG 300 to PEG 1450 comprises, without limitation, about 1:1 to about 1:10, about 1:1 to about 1:5, about 1:1 to about 1:3, about 1:1 to about 1:2, about 1:2 to about 1:3, about 1:1.25 to about 1:1.75, or about 1:1.5, 1:5, 1:10, 1:20, 1:25, 1:30, 1:40, 1:50, 1:60, 1:70, 1:75, 1:80, 1:90, 1:95, 1:99 and the like.

In an embodiment, the composition comprises sufficient quantities of the excipient or carrier to bring the volume of the composition up to a desired amount, such as 1 mL, 10 mL, 25 mL and the like.

The viscosity of the composition of the invention can be selected according to the desired treatment using conventional pharmaceutical compounding techniques and the pharmaceutical carriers and excipients described herein. In an embodiment, the viscosity of the composition is up to about 10,000 centipoise (Cp). In another embodiment, the viscosity of the composition is from about 5,000 Cp to about 200,000 Cp, from about 5,000 Cp to about 15,000 Cp, from about 25,000 Cp to about 200,000 Cp, from about 25,000 to about 100,000 Cp, from about 40,000 Cp to about 70,000 Cp, from about 125,000 Cp to about 200,000 Cp, from about 1,000,000 Cp to about 10,000,000 Cp, from about 1,000,000 Cp to about 2,000,000 Cp, from about 5,000,000 Cp to about 10,000,000 Cp.

Preferably, the composition of the invention is both flowable and moldable such that the composition can be inserted or packed into a dental wound, molded to conform to the shape of the dental wound, and is resistant to being washed out of the dental wound by saliva or other biological fluid but is easily removable, if necessary, from the dental wound. In an embodiment, the composition is a gel, cream, paste, or ointment. In another embodiment, the composition is shear thinning (i.e., behaves like a fluid when worked or agitated but then settles into a nearly solid state at rest). In an aspect, the shear thinning composition is pseudoplastic or a bingham plastic. Applying a shear stress to a shear thinning composition of the invention, such as a gel, cream, paste, or ointment, reduces the viscosity of the composition such that the composition can be packed into the dental wound and molded to conform to the shape of the dental wound. Once the shear stress is removed, the composition returns to its resting viscosity. The resting viscosity of the composition is selected such that composition is a solid or semi-solid at rest. Examples of a solid or semi-solid state include without limitation a cream, gel, paste, or ointment.

In an embodiment, the composition of the invention is made into a formulation by mixing together one or more antibiotics, preferably three antibiotics, a radiopaque substance, and a pharmaceutically acceptable excipient. Optional ingredients, such as an anesthetic agent, analgesic agent, or combination thereof, can also be mixed into the formulation. In an aspect, the pharmaceutical excipient is made by diluting or otherwise forming the excipient to a particular predetermined volume. The antibiotics, in powder form, for example, are then weighed to predetermined amounts and mixed with the excipient, followed by the addition of a predetermined amount of the radiopaque substance. In an aspect, the formulation of the invention is made, without limitation, as an ointment, cream, liquid, suspension, paste or powder. In another aspect, the composition of the invention is formulated as an ointment, paste or powder for topical application to a wound site. In yet another aspect, the formulation of the invention is provided in syringes of 1 mL, 5 mL, 10 mL and the like, for single or multiple use. In an aspect, the formulation of the invention is dispensed in amber syringes or vials for protection from the effects of light or uv radiation.

In an embodiment, the composition or formulation of the present invention is used in a method to disinfect a dental wound site in a subject. In an aspect, the subject is a mammal. The mammal can be a human. In an aspect, the dental wound site is, without limitation, the site of a root canal treatment, a treatment for cavities, for apical periodontitis in immature teeth, during extraction, and the like. In an aspect, the composition of the invention, formulated as an ointment, cream, paste, powder, liquid, or suspension, for example, is dispensed from 1 mL syringes and applied directly to the wound site.

In an embodiment, the composition or formulation of the present invention is used as a root canal sealant or sealing agent in typical dental procedures, such as root canals, endodontic treatment, and the like. In an aspect, the composition or formulation is used to coat a solid or semisolid core filling material to fill the disinfected and cleaned root canal, following treatment for pulpal or periapical inflammation, for example. In an aspect, the composition of formulation, when mixed and used as a sealant, has sufficient tackiness to provide good adhesion or bond strength with the dentin walls of the root canal or cleaned pulpal space. In an aspect, the mean bond strength produced by the composition or formulation of the present invention is 0 to 1 MPa, 0 to 2 MPa, 0 to 3 MPa, 0 to 4 MPa, 0 to 5 MPa, 0 to 6 MPa, 0 to 7 MPa, and the like. In an aspect, the composition or formulation, when mixed and used as a sealant, must be capable of forming a hermetic seal on the apical end of the root canal treatment site to prevent reinfection as a result of apical leaking from the disinfected and cleaned pulpal space. The efficacy of the hermetic seal, as demonstrated by low or absent apical leakage may be assessed by methods known to those of skill in the art, such as, without limitation, leakage studies using methylene blue or India ink as a staining agent, for example. In an aspect, the composition or formulation of the invention reduces or eliminates apical leakage when used as a root canal sealant material.

In an embodiment, the composition or formulation of the invention is used as a root canal sealant or sealing agent that is radiopaque and therefore visible on a radiograph. This ensures proper placement of the apical filling material coated with the sealant during root canal treatment. In an aspect, the radiopacity of the composition or formulation of the present invention is obtained from incorporating a radiopaque substance into the formulation or composition. In an aspect, the radiopaque substance is iodoform.

In an embodiment, the composition or formulation of the invention used as a root canal sealant or sealing agent, if present in solid form, has small or fine particle size. This ensures effective mixing of the agent with excipients or other liquids used during dental treatment. In an aspect, the particle size of the mixture of antibiotics and radiopaque substance in the composition of the present invention ranges from 0 to 5 microns, 0 to 10 microns, 0 to 20 microns, 0 to 30 microns and the like. In an aspect, the particle size is no more than 50 microns.

In an embodiment, the composition or formulation of the present invention used as a root canal sealant or sealing agent maintains its volume or size during and after treatment, i.e. there is no shrinkage once the material has set. In order to prevent reinfection, the root canal filling material must fill the entire pulpal space after disinfection during treatment. In an aspect, the composition or formulation of the present invention is used to coat root canal or apical filling materials to prevent shrinkage of the filling material following treatment, i.e. when the material sets.

In an embodiment, the composition or formulation of the present invention provides disinfection and sealing properties during dental treatment or when used as a root canal sealant without altering the aesthetic appearance or color of the teeth. In an aspect, the composition or formulation of the present invention does not contain components or ingredients known to cause discoloration of the enamel during treatment.

In an embodiment, the composition or formulation of the present invention provides disinfectant and sealing properties during dental treatment by being bactericidal or bacteriostatic. In an aspect, the bactericidal or bacteriostatic properties of the composition or formulation of the present invention are obtained from incorporating a mixture of one or more antibiotics into the formulation or composition. In an aspect, the mixture of one or more antibiotics is a mixture of three antibiotics. In an aspect, the mixture of antibiotics include, without limitation, nitroimidazoles, cephalosporin, lincosamide fluoroquinolone and the like. In an aspect, the composition or formulation of the present invention includes metranidazole, clindamycin or cefaclor and ciprofloxacin as the antibiotic components to ensure bacteriostatic or bactericidal properties.

In an embodiment, the composition or formulation of the present invention sets slowly when mixed for use as a sealant, to avoid premature setting of the sealant or root-filling material before the entire pulpal space or root canal has been filled. Sealant materials will set more rapidly in the warm and humid environment of the apical or pulpal space than in bench top conditions, and therefore, it is necessary to select sealant materials that set slowly. However, materials that set too slowly interfere with complete root canal treatment, and may cause tissue irritation at the wound site. In an aspect, the composition or formulation of the present invention has setting time optimal for root canal treatment. In another aspect, the composition or formulation of the invention has a setting time of less than one hour, less than 2 hours, less than 5 hours, less than 10 hours, and the like. In yet another aspect, the setting time for the composition of the invention is up to 20 minutes, of between 5 minutes and 45 minutes, of between 5 minutes and 30 minutes, of between 5 minutes and 45 minutes, of between 5 minutes and 50 minutes, and the like.

In an embodiment, the composition or formulation of the present invention should be insoluble in tissue fluids when used as a root canal sealant or sealing agent. This is an important feature of an effective sealing agent, as the apical seal must remain intact even in contact with tissue fluids. Typical tissue fluids that come in contact with dental surfaces include saliva, water, blood, plasma and the like. In an aspect, the composition or formulation of the invention is insoluble in such fluids.

In an embodiment, the composition or formulation of the present invention is biocompatible, i.e. well tolerated by the periapical tissue. The material used as a root canal sealant or sealing agent must be non-immunogenic in the periapical or periradicular space, and should be non-mutagenic and non-carcinogenic.

In an embodiment, the composition or formulation of the present invention is soluble in commonly used dental solvents. This is important, as it may be necessary to remove the root canal filling material after treatment, or during a retreatment procedure. In an aspect, the composition or formulation of the present invention is soluble in solvents such as, without limitation, sodium chloride, sodium hypochlorite, chloroform, formamide, phenyl ethanol, hydrogen peroxide, isopropyl alcohol, xylene, eucalyptus oil, combinations or mixtures thereof, and the like.

In another aspect, the composition of the invention is provided as a kit containing the components of the composition. The kit can include instructions for preparing the composition from the components provided in the kit and instructions for using the composition to treat a dental wound or as a dental sealant. In an embodiment, the kit comprises one or more antibiotics and a radiopaque substance. In another embodiment, the kit comprises one or more antibiotics, a radiopaque substance, and a pharmaceutically acceptable carrier or excipient. The components of the kit can be in powder form, a solution form, or a suspension form. The pharmaceutically acceptable carrier or excipient component(s) can be provided without limitation in powder form, solution form, liquid, suspension, cream form, paste form, gel form, ointment form, and the like. The components of the kit can be packaged in syringes, vials, ampoules, and the like. In an embodiment, the components can be mixed immediately prior to treatment to form the composition. The mixed components are then applied directly to the dental wound site. In an embodiment, the components can be mixed to form the composition and then stored for a period of time prior to use. Storage can be at ambient conditions, under refrigerated conditions, or under freezing conditions. In an embodiment, the composition once mixed is stable for up to one year. In another embodiment, the composition once mixed is stable for up to six months. In yet another embodiment, the composition once mixed is stable for up to three months. In an embodiment, the composition once mixed is stable for at least one year. In another embodiment, the composition once mixed is stable for at least six months. In yet another embodiment, the composition once mixed is stable for at least 3 months.

### EXAMPLES

The present invention may be better understood with reference to the following examples. These examples are intended to be representative of specific embodiments of the invention, and are not intended as limiting the scope of the invention.

### Example 1

This example demonstrates the preparation of a composition and formulation of the invention.

3.1 g of PEG 300 was melted in a beaker, and the temperature was held at 60° C. 7.1 mL of PEG 1450 was then added and stirred at low speed for a few minutes. 0.251 g of ciprofloxacin and 0.75 g of metranidazole were added to a mortar along with the contents of three (3) capsules of cefaclor (total weight 0.75 g) and crushed together. The required weight of the PEG bases was taken in a mortar, and allowed to congeal to an ointment consistency. The mixture of antibiotics was then added to the PEG mixture and triturated. The mixture was then run through an ointment mill twice to form the final composition and then the composition was dispensed into 1 mL amber syringes for topical application. This amount of the formulation is sufficient for 10 dental treatments. The final composition was found to be stable for three months under refrigerated conditions (2° C. to 8° C.). FIG. 1 shows a radiograph of a sample of the composition which demonstrates the radiopacity of the composition.

### Example 2

An embodiment of the composition of the invention includes the following components:

| Component | wt % |
|---|---|
| Nitroimidazole | 2-8 |
| Cephalosporin | 0.4-3.2 |
| Lincosamide or | 2-8 |
| fluoroquinolone | |
| Iodoform | 2-8 |
| Carrier and/or excipient | balance |

An embodiment of the composition of the invention includes the following components:

| Component | wt % |
|---|---|
| Nitroimidazole | 4-6 |
| Cephalosporin | 0.8-2.4 |
| Lincosamide or | 4-6 |
| fluoroquinolone | |
| Iodoform | 4-6 |
| Carrier and/or excipient | balance |

An embodiment of the composition of the invention includes the following components:

| Component | wt % |
|---|---|
| Nitroimidazole | 5 |
| Cephalosporin | 2 |
| Lincosamide or | 5 |
| fluoroquinolone | |
| Iodoform | 5 |
| Carrier and/or excipient | balance |

The nitroimidazole is ametranidazole, tinidazole, or nimorazole. The cephalosporin is cefaclor, cefonicid, cefprozil, cefuroxime, cefuzonam, cefmetazole, cefoxitin, or cefotetan. The fluoroquinolone is ciprofloxacin, moxifloxacin, or levofloxacin. The lincosamide is lincomycin or clindamycin.

### Example 3

An embodiment of the composition of the invention includes the following components:

| Component | wt % |
|---|---|
| Metronidazole | 2-8 |
| Ciprofloxacin | 0.4-3.2 |
| Cefaclor or clindamycin | 2-8 |
| Iodoform | 2-8 |
| Carrier and/or excipient | balance |

An embodiment of the composition of the invention includes the following components:

| Component | wt % |
|---|---|
| Metronidazole | 4-6 |
| Ciprofloxacin | 0.8-2.4 |
| Cefaclor or clindamycin | 4-6 |
| Iodoform | 4-6 |
| Carrier and/or excipient | balance |

An embodiment of the composition of the invention includes the following components:

| Component | wt % |
|---|---|
| Metronidazole | 5 |
| Ciprofloxacin | 2 |
| Cefaclor or clindamycin | 5 |
| Iodoform | 5 |
| Carrier and/or excipient | balance |

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

The above specification, examples and data provide a complete description of the manufacture and use of the compositions of the invention. The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention without following the example embodiments and applications illustrated and described herein, and without departing from the true spirit and scope of the present invention, which is set forth in the following claims.

What is claimed is:

1. A composition for disinfecting a dental wound site, comprising:
an active ingredient comprising one or more antibiotics;
a radiopaque substance; and
a pharmaceutically acceptable carrier or excipient.

2. The composition of claim 1, wherein the active ingredient is a mixture of one or more antibiotics, the mixture comprising a nitroimidazole antibiotic, a fluoroquinolone antibiotic, and a cephalosporin or a lincosamide antibiotic.

3. The composition of claim 2, wherein the nitroimidazole antibiotic comprises metronidazole, tinidazole, or nimorazole.

4. The composition of claim 2, wherein the fluoroquinolone antibiotic comprises ciprofloxacin, moxifloxacin, or levofloxacin.

5. The composition of claim 2, wherein the cephalosporin antibiotic comprises cefaclor, cefonicid, cefprozil, cefuroxime, cefuzonam, cefmetazole, cefoxitin, or cefotetan.

6. The composition of claim 2, wherein the lincosamide antibiotic comprises lincomycin or clindamycin.

7. The composition of claim 1, wherein the radiopaque substance comprises iodoform.

8. The composition of claim 1, wherein the pharmaceutically acceptable carrier or excipient comprises polyethylene glycol (PEG).

9. The composition of claim 8, wherein the pharmaceutically acceptable carrier or excipient comprises a ratio of about 3:1 to about 2:1 PEG 1450 to PEG 300.

10. The composition of claim 1, wherein the composition comprises:
about 0.5 to about 30 wt % nitroimidazole,
about 0.5 to about 10 wt % quinolone,
about 0.5 to about 30 wt % cephalosporin or lincosamide, and
about 0.5 to about 30 wt % iodoform.

11. The composition of claim 1, wherein the composition is a gel, cream, ointment, or paste.

12. The composition of claim 1, wherein the composition is in a container and the container comprises a syringe, vial, or ampoule.

13. The composition of claim 1, further comprising an anesthetic agent or an analgesic agent.

14. A method of disinfecting a dental wound in a subject, comprising administering the composition of claim 1 to the dental wound.

15. The method of claim 14, wherein the subject is human.

16. The method of claim 14, wherein the dental wound is associated with a root canal, caries, apical periodontitis, or pulpal necrosis.

17. The method of claim 14, further comprising radioimaging the dental wound after administration of the composition to confirm proper administration of the composition to the dental wound.

18. A kit for disinfecting a dental wound, comprising:
an active ingredient comprising one or more antibiotics;
a radiopaque substance;
and instructions for preparing a composition comprising the one or more antibiotics and the radiopaque substance to treat a dental wound.

19. The kit of claim 18, further comprising a pharmaceutical carrier or excipient.

20. The kit of claim 18, wherein the one or more antibiotics comprises a nitroimidazole antibiotic, a fluoroquinolone antibiotic, a cephalosporin antibiotic or a lincosamide antibiotic.

21. The kit of claim 18, wherein the one or more antibiotics comprises metronidazole, ciprofloxacin, and cefaclor or clindamycin.

22. The kit of claim 18, wherein the radiopaque substance comprises iodoform.

23. A composition for sealing the apical surface of a tooth following dental treatment, comprising:
an active ingredient comprising one or more antibiotics;
a radiopaque substance; and
a pharmaceutically acceptable carrier or excipient.

24. The composition of claim 23, further comprising an anesthetic agent or an analgesic agent.

25. The composition of claim 23, wherein the composition comprises:
about 0.5 to about 30 wt % of a nitroimidazole;
about 0.5 to about 10 wt % of a quinolone;
about 0.5 to about 30 wt % of a cephalosporin or lincosamide; and
about 0.5 to about 30 wt % of iodoform.
